(19) United States
(12) Patent Application Publication
(10) Pub. No.: US 2020/0390557 A1
(43) Pub. Date: Dec. 17, 2020

(54) CENTROMEDULLARY FIXATION STEM FOR JOINT PROSTHESIS

(71) Applicant: GILES, Caluire-Et-Cuire (FR)

(72) Inventors: Daniel Noyer, Luzinay (FR); Philippe Bauchu, Lyon (FR); Alain Cypres, Saint-Nizier-Sous-Charlieu (FR); Arnaud Fiquet, Caluire-Et-Cuire (FR); Christophe Roy, Chatuzange-Le-Goubet (FR); Olivier Bonnard, Meyzieu (FR); Philippe Girardin, Lezigneux (FR); Bertrand Seutin, La Batie-Rolland (FR); Gualter Vaz, Lyon (FR); Philip John Roberts, Cheshire (GB)

(21) Appl. No.: 16/509,990

(22) Filed: Jul. 12, 2019

(30) Foreign Application Priority Data
Jun. 11, 2019 (FR) 1906170

(51) Int. Cl. A61F 2/36; A61F 2/38; A61F 2/40; A61F 2/30

(52) U.S. Cl. CPC A61F 2/3601; A61F 2/367; A61F 2/3676; A61F 2/38; A61F 2002/3038; A61F 2/30749; A61F 2002/3674; A61F 2002/30354; A61F 2/40

(57) ABSTRACT

An anchoring stem for a joint prosthesis with a centromedullary fixation includes a metaphyseal-diaphyseal (M-D) portion intended to be inserted into the medullary canal of the long bone of the joint to be prosthesized. It receives an epiphyseal-diaphyseal (E-D) portion, having its upper portion protruding from the considered bone and intended to receive in turn an articular element. The M-D portion and the E-D portion are independent from each other but may be fastened to each other. The E-D portion is received within the M-D portion along a direction parallel or substantially parallel to the main dimension of the M-D portion, along a length of cooperation of the portions with each other such that the ratio of the length of reception of the E-D portion within the M-D portion to the total length of the M-D portion is in the range from 0.5 to 0.85. The M-D portion is provided with means capable of forbidding any rotation or angular displacement of the E-D portion once the latter has been received within the M-D portion.

### TECHNOLOGICAL FIELD

The disclosure pertains to the field of joint prostheses implementing a centromedullary fixation, and particularly an anchoring stem.

It more particularly concerns a femoral stem for a hip prosthesis, but also any other type of joint prosthesis with a centromedullary fixation, such as for example knee prostheses, shoulder prostheses, etc.

### BACKGROUND

Many types of total hip prostheses are known to date. Such prostheses conventionally comprise a femoral stem implanted in the femur, by impaction or cementation in the medullary canal of the concerned femur, and an acetabulum implanted in the pelvic bone. The end of the femoral stem is conventionally formed by a neck, receiving a head or ball having a generally spherical shape, engaged into said acetabulum, or in the absence thereof, into the acetabular cavity. Conventionally, an insert, mobile or not, conventionally made of polyethylene or of ceramic, capable of favoring the rotation of the ball or spherical head therein, and thus the general movement of the joint, is positioned in the acetabulum.

Such a head or ball is most often affixed to the neck of the stem by a taper, typically a Morse taper, having said head affixed at the level thereof by wedging of said head. Although, in principle, such prostheses have allowed a significant progress with respect to prior prostheses, a number of difficulties however still arise.

Among these, one of the difficulties is inherent to the management of the final neck length, comprising the neck and the head, as an extension of the metaphyseal portion of the stem. The head may be more or less covering on the taper to manage the necessary final length according to the situation. The area of the Morse taper which protrudes from the head or from the ball bears against the edge at the level of the polyethylene or ceramic insert, affixed or not in the acetabulum, particularly during angular motions of the joint, which in the long term wears the opposite material and, accordingly, generates debris or fractures or notches that may a rejection of said prosthesis or complications in the long term, requiring replacing said prosthesis.

As a variation, it has been provided to fit the articulation head or ball with a skirt at its base, that is, that the level of its area of cooperation with the Morse taper. However, such a solution is not satisfactory since the lower edge of the skirt, by increasing the diameter of the neck, precociously bears against the edge of the polyethylene or ceramic insert and, in the long term, a wearing of the mobile or fixed insert can be observed, with the same consequences as those previously discussed.

It has already been provided to use a femoral stem provided with a modular neck. In other words, the neck is placed on the upper end of the metaphyseal portion of the stem and is affixed thereto, particularly by taper wedging or by screwing. In addition to the relatively touchy implementation of such a stem, a number of breakages have been observed at level of the nesting of the neck on the upper portion of the metaphyseal portion of the stem, particularly due to the lever effect exerted at this level when the patient bears on the joint.

Another issue of such femoral stems concerns the need for the practitioner to have a wide range stock, to respond to the patient's anatomy and pathology, in terms of length and angulation of the neck, such as the valgus or varus angulation, for example, and of centromedullary bulk, particularly in the cases of a replacement or of a reconstruction, the three parameters being unconnected.

Thereby, such a significant stock requires being regularly renewed and accordingly generates a financially non-negligible investment and is further not necessarily present in the operating block at the necessary time.

The object of the presently described embodiments is to overcome all these different disadvantages.

### SUMMARY OF THE DISCLOSURE

Thus, the disclosed embodiments aim at an anchoring stem for a joint prosthesis with a centromedullary fixation, comprising a metaphyseal-diaphyseal portion intended to be inserted into the medullary canal of the long bone of the joint to be prosthesized, and, for example, of the femur for the hip joint, said metaphyseal-diaphyseal portion receiving an epiphyseal-diaphyseal portion, having its upper portion protruding from said long bone, and particularly from the medullary shaft, and intended to receive an articular element, in which stem the metaphyseal-diaphyseal portion and the epiphyseal-diaphyseal portion are independent from each other but may be fastened to each other.

According to embodiments:

the epiphyseal-diaphyseal portion is received within the metaphyseal-diaphyseal portion along a direction parallel or substantially parallel to the main dimension of said metaphyseal-diaphyseal portion, along a length of cooperation of said portions with each other such that the ratio of the length of reception of the epiphyseal-diaphyseal portion within the metaphyseal-diaphyseal portion to the total length of the metaphyseal-diaphyseal portion is in the range from 0.5 to 0.85;

the metaphyseal-diaphyseal portion is provided with means capable of forbidding any rotation or angular displacement of the epiphyseal-diaphyseal portion once the latter has been received within the metaphyseal-diaphyseal portion.

In other words, instead of arranging, as for example in the case of a so-called modular hip prosthesis, a femoral stem formed of elements assembled to one another in the upper area of the actual stem, and more exactly of the metaphyseal portion on the diaphyseal portion, particularly by means of a screw or the like, that is, by a proximodistal assembly, providing a fastening along the diaphyseal axis of the femur, that is, along the axis of transfer of the stress exerted on said femur, to take advantage of an area of mechanical cooperation between the two elements forming the upper stem, first enabling to have an adaptable epiphyseal-diaphyseal portion capable of having all the possible variations necessary for the practitioner according to the encountered pathology and anatomy, without generating the risk of breaking the neck (in the case of a hip prosthesis) or the end portion of said epiphyseal-diaphyseal portion due to the one-piece character of this portion, and, second, promoting the mechanical resistance necessary for the general prosthesis thus reconstructed by favoring the conditions of a cold welding of the two concerned portions, and thus having a monoblock final component. Such an optimization is further reinforced by the length of cooperation of the two portions forming the stem, since, provided to respect the previously-mentioned ratio, the stress exerted on the stem thus formed is more uniformly distributed, and accordingly the risks of breaking said stem are decreased.

It has thus be observed that if the previously-mentioned ratio is smaller than 0.5, the length of cooperation of the stem portions with each other is insufficient to reach the desired result, that is, the uniformization of the stress exerted on the stem.

Conversely, if this ratio is greater than 0.85, no real significant results in terms of mechanical resistance of the stem are obtained, and the forming of the metaphyseal-diaphyseal portion is thus made more complicated.

The fastening of the epiphyseal-diaphyseal portion within the metaphyseal-diaphyseal portion is performed by mechanical wedging or by cold welding. For this purpose, an intraoperative forming of the actual stem, that is, outside of the patient, can be envisaged. Thereby, all problems of contamination of the connectors by liquids such as blood, for example, are avoided, the practitioner selecting the epiphyseal-diaphyseal and metaphyseal-diaphyseal portion best adapted to the morphology of the medullary canal of the bone to be prosthesized, and this, independently from each other. The fastening of the two portions may for example be performed by means of a press which is extremely simple to form and to implement in a sterile environment.

The means capable of forbidding any rotation or angular displacement of the epiphyseal-diaphyseal portion once the latter has been received within the metaphyseal-diaphyseal portion are formed of wings or fins extending from a surface, particularly in the upper area of the metaphyseal-diaphyseal portion.

According to an advantageous embodiment, the cooperation between the epiphyseal-diaphyseal portion and the metaphyseal-diaphyseal portion is achieved by a dovetail-type assembly, the epiphyseal-diaphyseal portion being provided on a surface with a protrusion, for example, linear, extending along its entire height, said protrusion being capable of cooperating with a recess of complementary shape formed within the metaphyseal-diaphyseal portion. Such a cooperation mode may be inverted, that is, the protrusion may be positioned on the metaphyseal-diaphyseal portion, while the complementary recess is located within the epiphyseal-diaphyseal portion.

Typically, the cross-section of this cooperation is circular or substantially circular, but may be trapezoidal or have any other shape allowing such a fastening beneficial to a cold welding of the two portions in question.

### BRIEF DESCRIPTION OF THE DRAWINGS

The way in which the embodiments may be implemented and the resulting advantages will better appear from the following non-limiting embodiments, in relation with the accompanying drawings, and in relation with a femoral stem for a hip prosthesis.

FIG. 1 is a simplified perspective representation of a first embodiment.

FIG. 2 is a simplified representation in lateral view and in disassembled mode of the femoral stem of FIG. 1.

FIG. 3 is a simplified three-quarters front perspective representation of the metaphyseal-diaphyseal portion of the femoral stem of FIG. 1.

FIG. 4 is a lateral view of a second embodiment of the femoral stem.

FIG. 5 is a simplified representation in lateral view and in disassembled mode of the femoral stem of FIG. 4.

FIG. 6 is a simplified three-quarters front perspective representation of the metaphyseal-diaphyseal portion of the femoral stem of FIG. 4.

FIG. 7 schematically illustrates another fastening mode for the femoral stem in assembled mode.

FIG. 8 is a view of the femoral prosthesis of FIG. 7 in exploded view.

FIG. 9 schematically illustrates different variations of the metaphyseal-diaphyseal portion for a same epiphyseal-diaphyseal portion of the femoral stem.

FIG. 10 schematically illustrates different variations of the epiphyseal-diaphyseal portion for a same metaphyseal-diaphyseal portion of the femoral stem.

### DETAILED DESCRIPTION

Although the following description is more particularly directed towards a stem for a femoral hip prosthesis, it should be understood that the disclosed embodiments aim at applying to any type of joint prosthesis inducing a centromedullary fixation of a stem, and particularly a shoulder prosthesis, a knee prosthesis, etc.

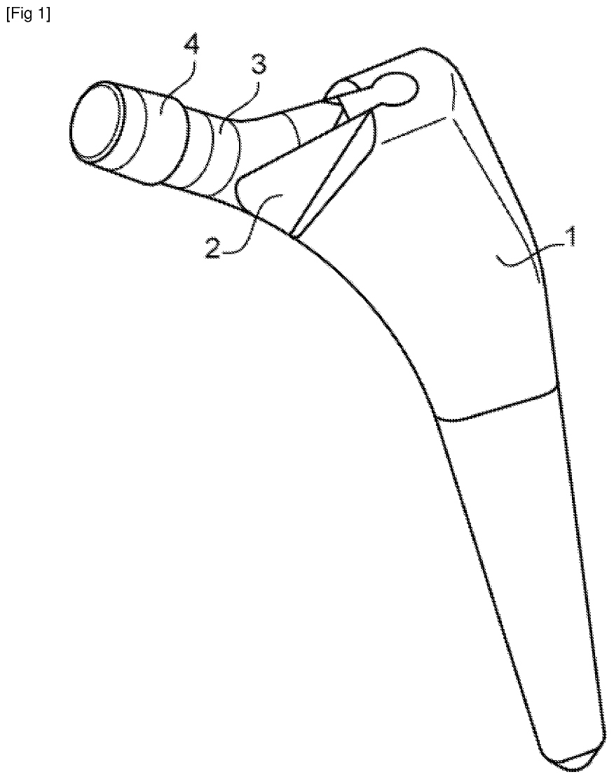

A first embodiment of a stem for a femoral hip prosthesis has thus been shown in relation with FIGS. 1 to 3.

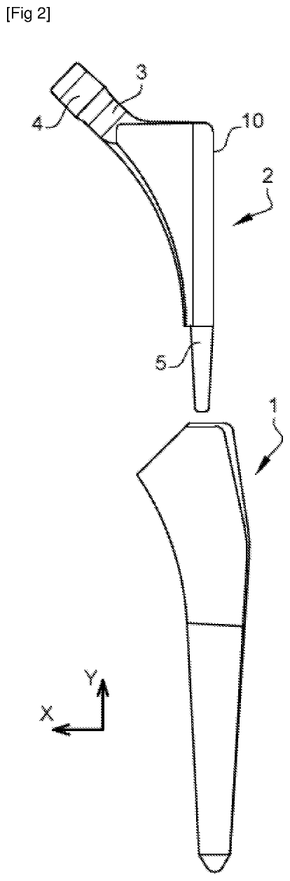

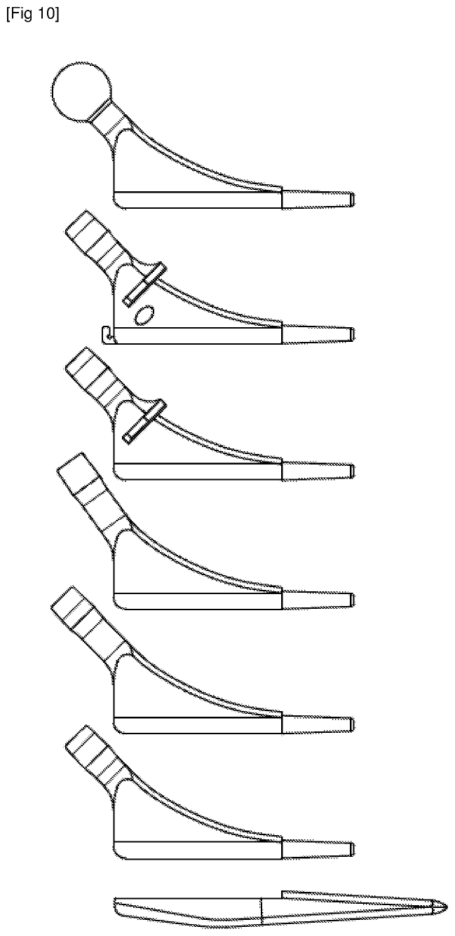

The stem is basically formed of two different portions intended to be fastened to each other, respectively a metaphyseal-diaphyseal portion (1) and an epiphyseal-diaphyseal portion (2) which can be better observed in FIG. 2, outside of the wound. The assembly, previously fastened, preferably, on a sterile table and outside of the patient, is intended to be introduced according to conventional techniques into the medullary canal of the femur, after a bone preparation by the surgeon, particularly by means of scrapers. Said epiphyseal-diaphyseal portion (2) has by construction a prosthetic neck (3) provided at its free end with a Morse taper (4) having a head or ball (11), itself intended to be received into the acetabular cavity of the considered joint or into an acetabulum or a cup, previously affixed in said acetabular cavity, fitting thereon by wedging. However, said head may also be machined in one piece on the epiphyseal-diaphyseal portion (see FIG. 10), and this, in known fashion.

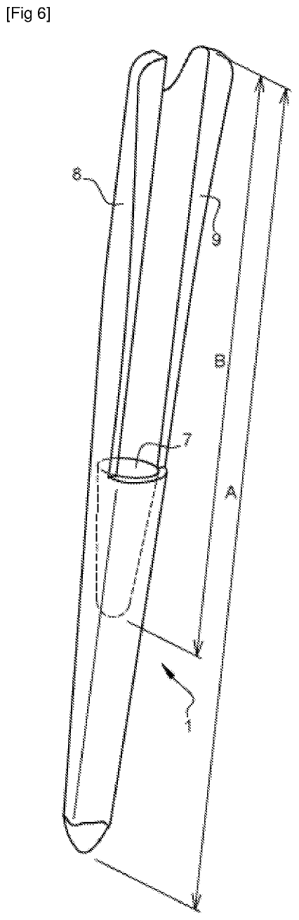

According to an essential feature of the invention, the cooperation of the epiphyseal-diaphyseal portion (2) with the metaphyseal-diaphyseal portion (1) to form the actual stem, is achieved along the diaphyseal axis of the femur and along a cooperation length such that the ratio of insertion length B of the epiphyseal-diaphyseal portion (2) within the metaphyseal-diaphyseal portion to the total length A of the metaphyseal-diaphyseal portion is close to 0.6. This length ratio has been illustrated in FIG. 6.

By adopting such a cooperation mode, and in the case of the described example of a hip prosthesis, said portions (1, 2) forming the stem cooperate together along the medial-lateral plane of the considered joint, schematized by reference frame (X, Y) in FIG. 2.

According to an essential feature of the invention, once the epiphyseal-diaphyseal portion has been introduced within the metaphyseal-diaphyseal portion, it is rotationally locked with respect to the diaphyseal axis of the metaphyseal-diaphyseal portion. Such a locking results from the presence of lateral wings (8, 9) extending from one of the surfaces of said metaphyseal-diaphyseal portion, and in the case in point, extrados. Thereby, once the stem thus formed is in place within the medullary canal of the femur, it remains properly oriented given the desired angular displacement in the medial-lateral plane.

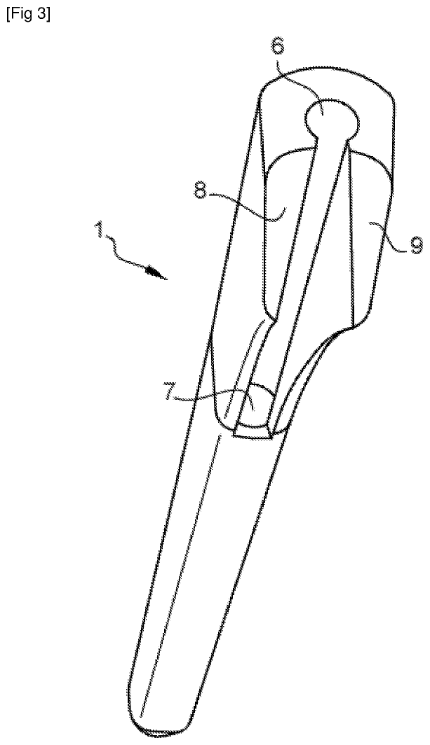

The cooperation between the epiphyseal-diaphyseal portion (2) and the metaphyseal-diaphyseal portion (1) is advantageously performed by mechanical wedging and/or by cold welding, after the introduction of a protrusion (5) extending substantially all along a lateral surface (10) of the epiphyseal-diaphyseal portion within a recess (6, 7) of complementary shape formed inside of the metaphyseal-diaphyseal portion (1). A dovetail-type assembly is typically used. For this purpose, and advantageously, the cross-section of the protruding portion (5) of the epiphyseal-diaphyseal portion is typically circular, as can for example be observed in FIG. 3.

Thereby, due on the one hand to the length of the area of cooperation between the epiphyseal-diaphyseal portion and the metaphyseal-diaphyseal portion (1) and, in the case in point, along substantially the entire height of the epiphyseal-diaphyseal portion (2), and on the other hand, to the reception and to the lateral hold of the epiphyseal-diaphyseal portion (2) between the upper lateral wings (8, 9) of the metaphyseal-diaphyseal portion, the stress distribution area is significantly increased with respect to modular femoral stems of the prior state of the art.

The epiphyseal-diaphyseal portion being monoblock, risks of breaking the prosthetic necks, as can be observed in said prior art devices, are thus drastically reduced.

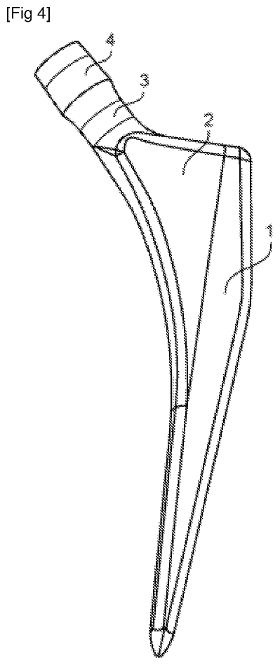

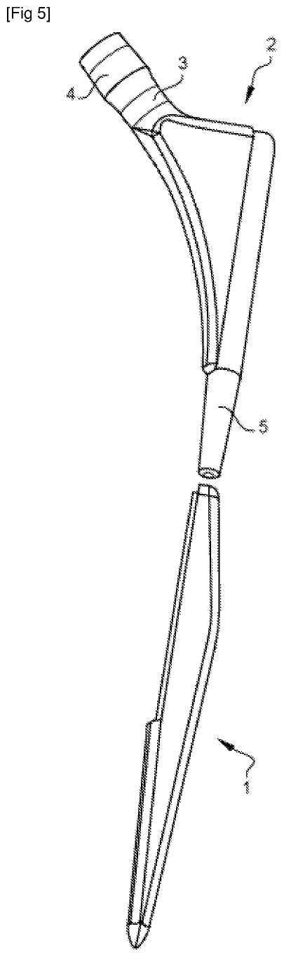

A second embodiment has been shown in relation with FIGS. 4 to 6. The principle underlying the second mode is strictly identical to that described in relation with FIGS. 1 to 3, except however for upper lateral wings which are smaller at the level of the diaphyseal portion. This embodiment is simpler to form, but does not modify the general principle underlying the general embodiments. FIG. 6 also shows the ratio of the respective lengths of the epiphyseal-diaphyseal and metaphyseal-diaphyseal portion, especially by showing the lower reception area of said epiphyseal-diaphyseal portion. Length B actually corresponds to the total height of the epiphyseal-diaphyseal portion.

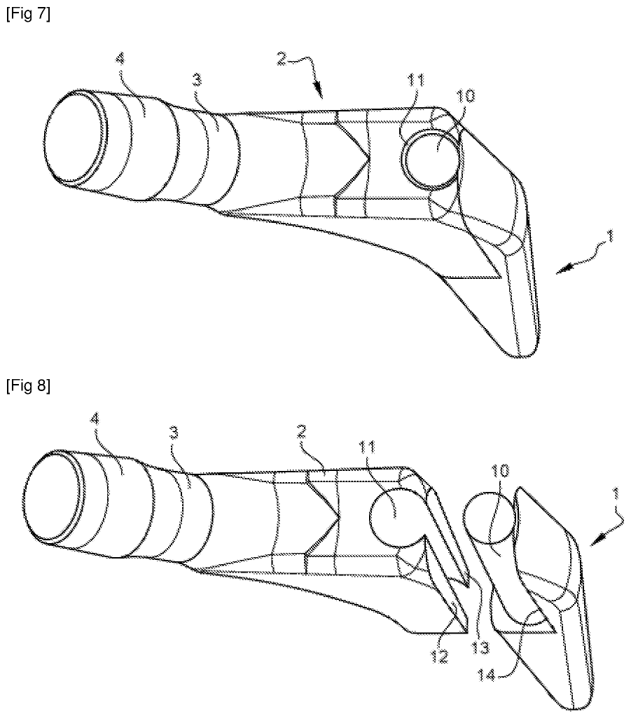

Another embodiment has been shown in relation with FIGS. 7 and 8. This mode is for the most part equivalent to the previous embodiment, with the difference that, however, the recess (11) is formed within the epiphyseal-diaphyseal portion (2), which is thus introduced on a protrusion (10) of complementary shape extending within the metaphyseal-diaphyseal portion (1). In this embodiment, the means capable of locking any rotation of the epiphyseal-diaphyseal portion once it has been fastened to the metaphyseal-diaphyseal portion (1) are formed of substantially planar surfaces (12, 13) extending parallel or substantially parallel to the recess (11), said surfaces cooperating with the inner surface (14) of the upper area of the metaphyseal-diaphyseal portion.

Due to the general principle underlying the present embodiments, it becomes possible for the practitioner to compose and to calibrate the femoral stem according to the patient's specific anatomy and pathology. He/she may in particular at leisure select the metaphyseal-diaphyseal portion of the stem, typically with a variable extrados according to the size to be implemented, and assemble thereto an epiphyseal-diaphyseal portion independent, particularly in terms of neck length, from the neck angle.

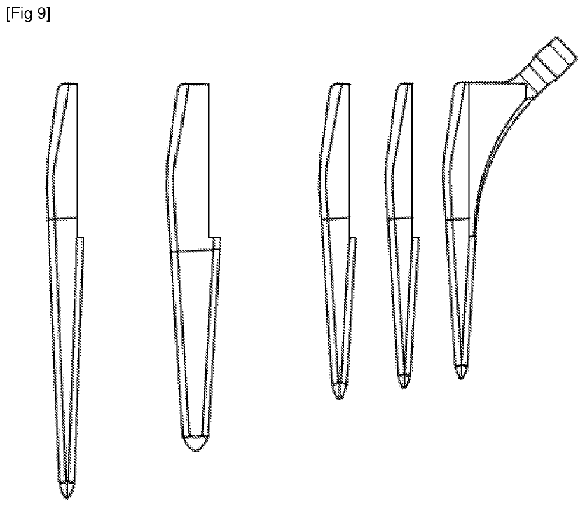

Such a modularity has in particular been illustrated at the level of FIGS. 9 and 10.

In other words, whatever the size of the extrados used (metaphyseal-diaphyseal portion), a plurality of neck lengths may be provided for the epiphyseal-diaphyseal portion, thereby eliminating the need for a wide range of femoral heads. Thereby, the practitioner can select the best adapted epiphyseal-diaphyseal portion so that the Morse taper, receiving the femoral ball, is entirely covered and non-overlapping, which, as above-targeted, then enables to do away with risks of wearing of the polyethylene insert, particularly in the context of dual-mobility hip prostheses.

The advantage of the stem, which provides at the same time an adaptability, an adjustment, a composition, a calibration, and a dimension depending on the pathology and on the anatomy encountered by the practitioner, including intraoperatively, and not, as in the prior state of the art, only based on the X-rays previously obtained, thus appears.

In addition to such an optimized modularity, the described embodiments further enable to significantly decrease the necessary stock in the range of prostheses to be used.

1. An anchoring stem for a joint prosthesis with a centromedullary fixation, comprising a metaphyseal-diaphyseal portion configured for insertion into a medullary canal of a long bone of a joint to be prosthesized, and receiving an epiphyseal-diaphyseal portion, the epiphyseal-diaphyseal portion having an upper portion protruding from the long bone, and configured to receive in turn an articular element, and wherein the metaphyseal-diaphyseal portion and the epiphyseal-diaphyseal portion are independent from each other but fastenable to each other:
wherein the epiphyseal-diaphyseal portion is received into the metaphyseal-diaphyseal portion along a direction parallel to a main dimension of said metaphyseal-diaphyseal portion, along a length of cooperation of the epipheseal-diaphyseal and metaphyseal-diaphyseal portions with each other such that a ratio of a length of reception of the epiphyseal-diaphyseal portion within the metaphyseal-diaphyseal portion to a total length of the metaphyseal-diaphyseal portion is in the range from 0.5 to 0.85; and
wherein the metaphyseal-diaphyseal portion is provided with (i) wings or fins extending from a surface in an upper area of the metaphyseal-diaphyseal portion or (ii) substantially planar surfaces cooperating with an inner surface of an upper portion of the metaphyseal-diaphyseal portion, once the epiphyseal-diaphyseal portion has been received within the metaphyseal-diaphyseal portion.

2. The anchoring stem for a joint prosthesis with a centromedullary fixation of claim 1, wherein the epiphyseal-diaphyseal portion is fastenable to the metaphyseal-diaphyseal portion by mechanical wedging or by cold welding.

3. The anchoring stem for a joint prosthesis with a centromedullary fixation of claim 1, wherein the means capable of forbidding any rotation or angular displacement of the epiphyseal-diaphyseal portion once the latter has been received within the metaphyseal-diaphyseal portion are formed of wings or fins extending from a surface in an upper area of the metaphyseal-diaphyseal portion.

4. The anchoring stem for a joint prosthesis with a centromedullary fixation of claim 1, wherein the means capable of forbidding any rotation or angular displacement of the epiphyseal-diaphyseal portion once the latter has been received within the metaphyseal-diaphyseal portion are formed of substantially planar surfaces cooperating with an inner surface of an upper portion of the metaphyseal-diaphyseal portion.

5. The anchoring stem for a joint prosthesis with a centromedullary fixation of claim 1, wherein the cooperation between the epiphyseal-diaphyseal portion and the metaphyseal-diaphyseal portion is achieved by a dovetail-type assembly, one of said portions being provided with a protrusion capable of cooperating with a recess of complementary shape formed within the other portion.

6. The anchoring stem for a joint prosthesis with a centromedullary fixation of claim 5, wherein a cross-section of the protrusion is circular, trapezoidal, or has any other shape enabling to fasten the two epiphyseal-diaphyseal and metaphyseal-diaphyseal portions.
